(19) United States
(12) Patent Application Publication
(10) Pub. No.: US 2005/0220716 A1
(43) Pub. Date: Oct. 6, 2005

(54) Drug delivery assembly

(76) Inventor: Sandrine Mireille Paulette Cuney, Cambridge (GB)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/513,791

(22) PCT Filed: May 16, 2003
(86) PCT No.: PCT/EP03/05192

§ 371 Date: May 27, 2005

(30) Foreign Application Priority Data
May 22, 2002 (GB) 0211753.9

(51) Int. Cl. A61L 9/04; A61M 11/00

(52) U.S. Cl. 424/45; 128/200.23

(57) ABSTRACT

This invention relates to a drug delivery assembly which includes a pressurised container (10) holding a drug formulation with a propellant, the container being disposed within a sealed enclosure (12) forming an overwrap or secondary packaging comprising a gas adsorbing material consisting of a microporous zeolite having a pore opening size less than 20 Å.

### FIELD OF THE INVENTION

This invention relates to a drug delivery assembly which includes a pressurised container holding a drug formulation with a propellant, the container being disposed within a sealed enclosure forming an overwrap or secondary packaging.

### BACKGROUND OF THE INVENTION

An example of such a container is a pressurised metered dose inhaler (p-MDI) where the vapour pressure of the propellant is used to deliver precisely metered doses of the drug formulation through a metering valve forming the container outlet. For many years p-MDIs have used chlorofluorocarbons (CFCs) as propellants. However, due to growing awareness that CFCs contribute to ozone depletion, manufacturers have searched for alternative propellants which are more environmentally friendly and fulfil propellant requirements.

Only hydrofluorocarbons (HFCs) such as hydrofluoroalkanes (HFAs) and specifically 1,1,1,2-tetrafluoroethane (HFA134a) and 1,1,1,2,3,3,3-heptafluoropropane (HFA227) have emerged as suitable for pharmaceutical use and the change from CFC to HFA has triggered new drug formulation development.

One drawback of HFCs is that with much lower boiling points than CFCs, they tend to leak from the p-MDIs through the plastic materials of the metering valve. Any propellant leakage causes a problem for p-MDIs that require a secondary packaging (typically to prevent either moisture ingress or particle contamination), as the leakage creates an overpressure in the secondary packaging:
if the secondary packaging is an impermeable flexible enclosure, the latter inflates and/or may burst;
if the secondary packaging is semi-rigid enclosure (such as a blister pack) and impermeable, it may burst.

Furthermore, in the particular case of p-MDI formulations containing a co-solvent such as ethanol, the overpressure problem in the enclosure is accompanied by the undesirable release into the enclosure of strong co-solvent odours. The overpressure in the enclosure and the release of co-solvent odours on opening of the enclosure are unacceptable for both patients and regulatory authorities. The invention aims to solve the problem of inflation of the enclosure due to propellant leakage. In its preferred form, the invention tackles the problem of co-solvent odour.

### PRIOR ART

Glaxo Group International patent application published under WO 00/37336 provides a flexible package for storing a pressurized container filled with a drug and a propellant, said package preventing ingression of water vapour and particulate matter while permitting egression of the propellant whereby shelf life of the drug is prolonged and performance of the drug and the propellant are maintained or increased.

The package is impermeable to water vapour and permeable to the propellant and further comprises means for absorbing moisture in the enclosed volume. The moisture absorbing material is preferably a silica gel desiccant sachet. Other materials include desiccants made from inorganic materials such as zeolites and aluminas.

WO 00/87392 relates to a flexible package or pouch further including a one-way valve to permit any propellant leaking from the pressurized container to egress from the pouch. The desiccant includes calcium sulfate, silica gel and casein/glycerol. A 4A molecular sieve is only generically cited among the other possible desiccant. There is no preference for this kind of desiccant over, for example, silica gel.

In WO 01/97888 the moisture absorbing material is located Within the pressurized container. The desiccant may be a nylon, silica gel, zeolite, alumina, bauxite, anhydrous calcium sulphate, activated bentonite clay, water absorbing clay, molecular sieve or combinations thereof.

WO 01/98175 relates to an apparatus wherein a substantially moisture-impermeable polymeric film is heat-shrinked onto at least a portion of the exterior of the device, the polymeric film comprising a first moisture absorbing material and a second moisture absorbing material being located within the pressurized container.

The absorbing material is a desiccant selected from the group consisting of nylon, silica gel, zeolite, alumina, bauxite, anhydrous calcium sulphate, activated bentonite clay, water absorbing clay, molecular sieve and combinations thereof.

WO 01/98176 describes an apparatus wherein the desiccant selected from the group consisting of nylon, silica gel, alumina, bauxite, anhydrous calcium sulphate, activated bentonite clay, a molecular sieve zeolite and combinations thereof, is in the form of a layer which adheres to the pouch.

### SUMMARY OF THE INVENTION

According to the invention a drug delivery assembly comprises:
a pressurised container holding a drug formulation with a propellant;
a sealed enclosure which surrounds the container and which is made of a moisture impermeable or substantially moisture impermeable material; and
a gas adsorbing material within the enclosure, the gas adsorbing material being a microporous zeolite having a pore opening size less than 20 Å, the gas adsorbing material being effective to adsorb propellant that might leak from the container into the enclosure.

The drug delivery assembly of the invention is effective and low-cost and may avoid the insertion of a one-spray valve in the enclosure.

The adsorption of leaked propellant by the gas adsorbing material (with the specified pore size) prevents inflation of the enclosure, where the latter is made from a flexible material. The enclosure may alternatively be made from a rigid or semi-rigid material.

The drug formulation within the container may be accompanied by a co-solvent, in which case the gas adsorbing material is preferably effective also to adsorb any leaked co-solvent, thereby avoiding unpleasant odours on opening of the enclosure.

The co-solvent is preferably an alcohol. The most preferred is ethanol.

The zeolite may be a natural mineral or may beta synthetically produced zeolite, commonly known as a molecular sieve. The size of the pores of the molecular sieve is critical for an effective adsorption of the propellant. In either case, the range of pore size is 4 Å to 20 Å, more preferably of 5 Å to 20 Å with a range of 8 Å to 15 Å being particularly favoured. The optimum pore size is 10 Å or substantially 10 Å, because this gives the best adsorption of propellant and co-solvent, where present.

As said before, the enclosure can be rigid, semirigid or flexible and it is preferably made from a flexible laminated multi-layer material, consisting of at least one heat sealable layer, at least one layer of a metal foil, and a protective layer. The material is impermeable to water vapour and can be in some cases at least partially permeable to a propellant and/or a cosolvent wherein the cosolvent is an alcohol and preferably ethanol. Such a three-layer laminate may have, for example, an outer protective layer (e.g. of polypropylene film), an intermediate layer of metal e.g. aluminium foil and a sealing layer (e.g. of polyethylene film).

Anyway, for the purposes of the invention the enclosure is preferably made of flexible packaging material or pouch. The material can be any material which is impervious to or substantially impervious to moisture and can be at least partially permeable to propellants such as HFA-134a and/or HFA-227.

### BRIEF DESCRIPTION OF THE DRAWINGS

A drug delivery assembly according to the invention will now be described, by way of example, with reference to the accompanying drawings, in which:

FIG. 1 illustrates the assembly,

FIG. 2 is a diagrammatic cross-sectional view on the line II-II of

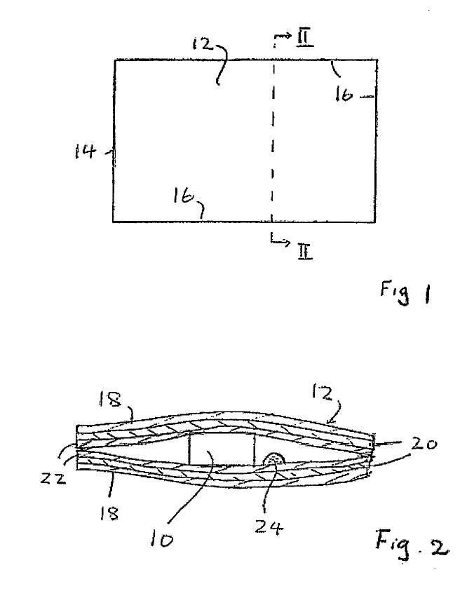

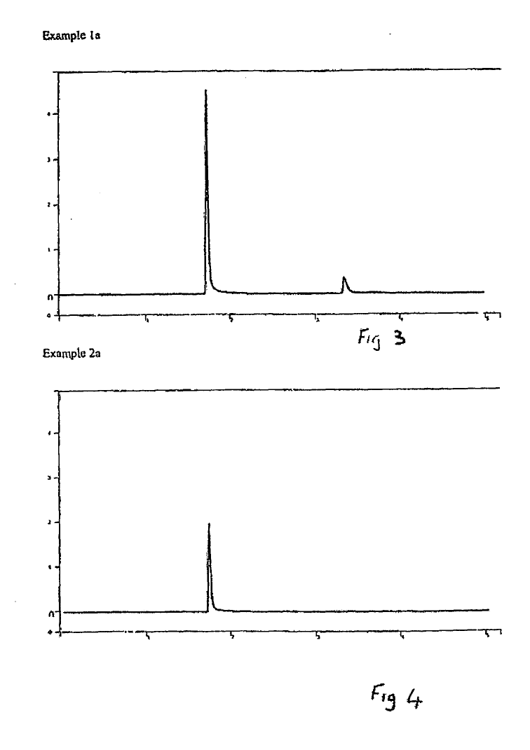

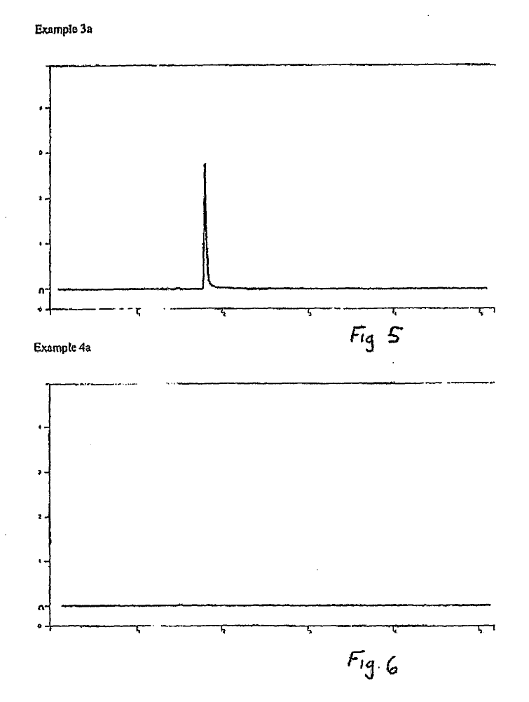

FIG. 1, and

FIGS. 3 to 9 are graphs and diagrams illustrating test results.

### DETAILED DESCRIPTION OF THE DRAWINGS

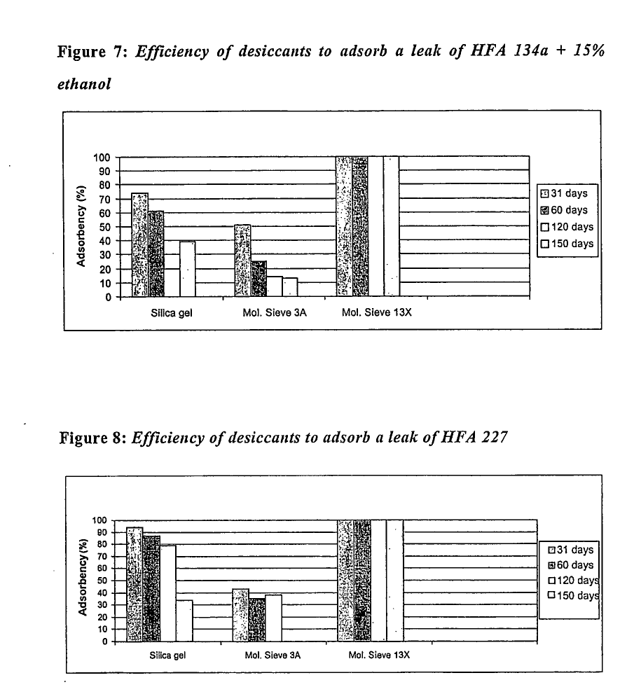

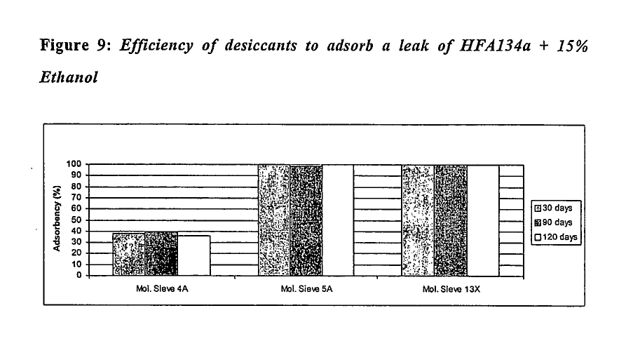

The drug delivery assembly shown in FIGS. 1 and 2 comprises a p-MDI 10, incorporating a drug formulation with an HFA propellant, the vapour pressure of which pressurises a container of the p-MDI 10 so that in use operation of an actuator releases a normally-closed valve to deliver metered doses of the drug formulation.

The p-MDI 10 is enclosed by an enclosure 12 forming a secondary packaging or overwrap. The enclosure 12 is made from a sheet of flexible material folded along a line 14 and sealed around the three remaining edges 16 so as to form a sealed pouch of generally rectangular shape. The flexible material of the enclosure is a three-layer laminate (FIG. 2) made up of an outer protective layer 18 of orientated polypropylene (OPP) having a thickness of 25 microns, an intermediate layer 20 of aluminium foil having a thickness of 9 microns and an inner sealing layer 22 of high density polyethylene (HDPE) having a thickness of 50 microns. The three-layer laminate material is substantially moisture impermeable, having a moisture vapour transmission rate below 0.1 g/m² per 24 h (measured according to ASTM E-398).

Within the sealed enclosure 12 is a body of microporous zeolite 24 having a pore opening size of 4 Å to 20 Å, the purpose of which is to adsorb any propellant which might leak from the p-MDI 10. Further, the zeolite 24 adsorbs any ethanol which is commonly used as a co-solvent for the drug formulation in the p-MDI. The adsorption of any leaking propellant or ethanol prevents both inflation of the enclosure 12 and a smell of ethanol on opening of the package prior to use of the p-MDI 10.

### DETAILED DESCRIPTION OF THE INVENTION

It has been found that a particular gas adsorbing material within a drug delivery assembly of the kind previously described, said gas adsorbing material consisting in a molecular sieve with a pore size comprised between 4 Å and 20 Å, preferably between 5 Å and 20 Å, more preferably between 8 Å and 15 Å, is effective to adsorb, besides moisture, the propellant and the co-solvent that might leak from the pressurized container into the enclosure in order to solve the problems of the overpressure in the enclosure and of the undesirable co-solvent odour on opening the enclosure.

The gas adsorbing material can be contained in a sachet placed in the enclosure. Alternatively the sachet can be loose in the pMDI or fixedly attached to them or be a part of an assembly attached to the pMDI.

The gas adsorbing material can be in the form of a layer, coating, lining or mesh and it can also adhere to the pouch.

A series of experiments has been carried out, where enclosures made out of impermeable flexible material containing a p-MDI (of the nature of the p-MDIs described previously in this document) and different materials with gas adsorbing properties have been stored at 40° C. and 75% RH for 30 days, 60 days, 90 days, 120 or 150 days.

Gas chromatography is the analytical method chosen to show the efficiency of the different substances to adsorb the leakage of HFA and ethanol.

In the Examples that follow, p-MDIs containing 12 ml of a mixture of HFA 134a and ethanol as a cosolvent or HFA 227 are used. The ratio propellant:cosolvent can be from 95%:5% to 80%:20%. In the examples the ratio is 85%:15%.

For all examples, the enclosure is a flexible pouch as described with reference to FIGS. 1 and 2.

Silica gel, molecular sieve 3A-EPG (pore size 3 Å), molecular sieve 4A (pore size 4 Å), molecular sieve 5A (pore size 5 Å), molecular sieve 13X-APG (pore size 10 Å) and activated alumina A201 are tested, in two different experimental sections, as a desiccant, in comparison with pouches without a gas adsorbing substance.

The quantities of gas adsorbing substances have been calculated according to the method reported in the following, using:
the average leakage rate of the p-MDIs, determined experimentally during stability trials at 40° C. and 75% RH
the adsorbing capacity of the substances, determined for water vapour by suppliers.
   Gas Adsorbing Substance Quantities: The quantities of desiccant placed in the different pouches have been calculated to provide enough desiccant or adsorbing capacity to adsorb:
The moisture permeating from the environment into the pouch: a desiccant adsorbs molecules by order of increasing size. Water vapour is the smallest molecule present in the pack and will therefore be adsorbed first.
The leak of HFA 134a +ethanol from the canister.
   We have evaluated that:
Water permeating through the pouch, over a six-month storage period at 40° C. and 75% RH is 0.265 g. This is based on a pouch size of 105×140mm and MVTR [Moisture Vapour Transmission Rate, i.e. the velocity by which the humidity permeates through a membrane (g/m²/day)] of 0.1 g/m².24 h
The amount of HFA 134a/ethanol leaking from a canister stored at 40° C. and 75% RH is 150 mg/year
We have assumed that the leak rate of canisters containing HFA 227 as a propellant is similar to the leak rate of canisters containing HFA 134a and ethanol
   Assuming that the capacity of desiccant for ethanol and propellant is similar to water capacity, the total amount of gas to be adsorbed over six month storage at 40° C. and 75% RH is 0.34 g

Prior to packaging and storage in controlled conditions, the weight of each p-MDI was recorded. Each p-MDI was then placed in a pouch with or without a gas adsorbing substance. Each pouch was then heat-sealed, and left for a given storage period.

During that period propellant and co-solvent leaked from the p-MDI into the pouch. This leakage resulted in a reduction of the overall weight of the p-MDI. Since the leakage was an ongoing, continuous process, the amount of weight loss of the p-MDIs increased with increasing storage times.

The leakage was greater for the p-MDIs containing HFA 134a than for those containing HFA227. This is because HFA134a has a lower boiling point than HFA 227: −26° C. for HFA 134a, −16° C. for HFA227. Pouch inflation is therefore a greater potential problem for the p-MDIs using HFA 134a propellant.

After the various storage period at 40° C. and 75% RH:
A sample of gas was taken from each Example and analysed by Gas Chromatography (GC), using a methodology developed by the applicants, which enables the separation of HFA 134a and ethanol.
For each example, the pouch was opened, the p-MDI removed from its enclosure and weighed to calculate its weight loss
For some samples the operator assessed ethanol odour upon pouch opening.

The GC method allows to separate HFA134a from ethanol. There is a linear relationship between the amount of HFA 134a, HFA 227 or ethanol injected in the column and the detector response.

One can therefore use GC traces to compare the efficiency of a gas adsorbing substance to adsorb HFA or a mixture HFA/ethanol, using the following formula:

$${A}_{corrected} = ( 1 - \frac{( {S}_{HFA . i} + {S}_{Eth . i} )}{( {S}_{HFA . ref} + {S}_{Eth . ref} )} \times \frac{{L}_{ref}}{{L}_{i}} ) \times 100 where \text{:}$$

A_{corrected} is the corrected efficiency of desiccant in Sample i
Lᵢ is the weight loss of the canister in sample i
L_{ref} is the weight loss of the canister in the sample containing no desiccant.
S_{HFA.i} is the area of the GC peak characteristic of HFA for the gas sample taken from sample i
S_{Eth..i} is the area of the GC peak characteristic of Ethanol for the gas sample taken from sample i
S_{HFA.ref} is the area of the GC peak characteristic of HFA for the gas sample taken from the canister containing no desiccant
SEth..ref is the area of the GC peak characteristic of Ethanol for the gas sample taken from the canister containing no desiccant.

The GC chromatograms for Examples 1a to 4a are presented in FIGS. 3 to 6. These chromatograms were obtained after 31 days storage.

FIGS. 7-9 show the efficiency of different gas adsorbing substances over time to adsorb respectively a leak of HFA+15% ethanol and a leak of HFA 227.

The GC trace of Example 1a exhibits two peaks: the first one (at 1.7 min) is characteristic of HFA 134a; the second one (at 3.3 min) is characteristic of ethanol. When opening the enclosure in Example 1a, the operator detects a strong ethanol smell.

The GC traces of the Examples 2a to 4a do not exhibit any peak characteristic of ethanol: all the gas adsorbing substances tested in these different Examples are efficient to adsorb ethanol. In addition, the operator did not detect any ethanol odour when enclosures are opened.

The different gas adsorbing substances tested are efficient to adsorb some of the HFA 134a leak, but this efficiency decreases over time, except for molecular sieves 5 Å and 13X, which keep their efficiency of adsorbing completely the HFA134a leak after 120 and 150 days respectively (FIGS. 7-9).

These results indicate that a molecular sieve of porous size of at least 4 Å, preferably at least 5 Å has a favourable adsorption isotherm in the test conditions for both ethanol and HFA 134a. As a result of complete HFA 134a adsorption, enclosure inflation is almost eliminated.

Furthermore, in order to evaluate the effectiveness of the drug delivery assembly of the invention, shelf-life tests were carried out upon a package which contained a pMDI containing formoterol fumarate as active ingredient, in solution in HFA 134a and ethanol.

Degradation products and water content of a formulation containing formoterol fumarate 6 mcg/50 μl were assessed initially and after 1.5, 3 and 6 months.

In this particular example the package contained molecular sieve 13X-APG desiccant. Unpouched and pouched with and without the desiccant pMDIs were compared.

It has been so demonstrated that the drug delivery assembly of the invention allows to reduce the moisture ingress into the pMDI and to improve the chemical stability of the drug product.

The assembly of the invention applies to any HFA composition comprising formoterol, its enantiomers or diastereoisomers, salts or solvates thereof, as active ingredient and, more generally, is particularly useful as a secondary packaging for pMDIs containing in the formulation active ingredients sensitive to water.

### EXAMPLES 1-14

The results obtained with pMDI containing 12 ml of a mixture of HFA 134a and ethanol or HFA 227 in the different experimental sections are shown in the following tables.

Weight losses of the pMDIs and leak adsorption for canisters containing the propellant with or without the cosolvent after storage in stressed conditions at 40° C. and 75% RH are reported.

Table 1a, 1b and 1c: Summary of the different examples

| | | | | | Gas |
|---|---|---|---|---|---|
| | | | | Gas adsorbing | adsorbing |
| Example | Storage | P-MDI content | | substance | substance |
| number | period | description | Enclosure description | description | weight (g) |
| Example 1a | 30 days | 85% HFA 134a + 15% | OPP (25 μm)/Aluminium foil | None | — |
| Example 1b | 60 days | ethanol | (9 μm)/HDPE (50 μm) | | |
| Example 1c | 120 days | | | | |
| Example 1d | 150 days | | | | |
| Example 2a | 30 days | | | Silica gel | 1.5 |
| Example 2b | 60 days | | | | |
| Example 2c | 120 days | | | | |
| Example 2d | 150 days | | | | |
| Example 3a | 30 days | | | Molecular Sieve | 2.2 |
| Example 3b | 60 days | | | 3A-EPG | |
| Example 3c | 120 days | | | | |
| Example 3d | 150 days | | | | |
| Example 4a | 30 days | | | Molecular Sieve | 1.8 |
| Example 4b | 60 days | | | 13X-APG | |
| Example 4c | 120 days | | | | |
| Example 4d | 150 days | | | | |
| Example 5a | 30 days | | | Activated | 1.1 |
| Example 5b | 60 days | | | alumina A201 | |
| Example 5c | 120 days | | | | |
| Example 6a | 30 days | HFA 227 | OPP(25 μm)/Aluminium foil | None | — |
| Example 6b | 60 days | | (9 μm)/HDPE (50 μm) | | |
| Example 6c | 120 days | | | | |
| Example 7a | 30 days | | | Silica gel | 1.5 |
| Example 7b | 60 days | | | | |
| Example 7c | 120 days | | | | |
| Example 7d | 150 days | | | | |
| Example 8a | 30 days | | | Molecular Sieve | 2.2 |
| Example 8b | 60 days | | | 3A-EPG | |
| Example 8c | 120 days | | | | |
| Example 8d | 150 days | | | | |
| Example 9a | 30 days | | | Molecular Sieve | 1.8 |
| Example 9b | 60 days | | | 13X-APG | |
| Example 9c | 120 days | | | | |
| Example 9d | 150 days | | | | |
| Example 10a | 30 days | | | Activated | 1.1 |
| Example 10b | 60 days | | | alumina A201 | |
| Example 10c | 120 days | | | | |
| Example 11a | 30 days | 85% HFA | OPP(25 μm)/Aluminium foil | Molecular Sieve | 2.5 |
| Example | 90 days | 134a + 15% | (9 μm)/HDPE (50 μm) | 4A | |
| 11b | | ethanol | | | |
| Example 11c | 120 days | | | | |
| Example 12a | 30 days | | | Molecular Sieve | 1.9 |
| Example | 90 days | | | 5A | |
| 12b | | | | | |
| Example 12c | 120 days | | | | |
| Example 13a | 30 days | HFA 227 | OPP(25 μm)/Aluminium foil | Molecular Sieve | 2.5 |
| Example | 90 days | | (9 μm)/HDPE (50 μm) | 4A | |
| 13b | | | | | |
| Example 13c | 120 days | | | | |
| Example 14a | 30 days | | | Molecular Sieve | 1.9 |
| Example | 90 days | | | 5A | |
| 14b | | | | | |
| Example 14c | 120 days | | | | |

OPP = Oriented PolyPropylene
HDPE = High Density PolyEthylene

TABLE 1d
Weight losses and leak adsorption for canisters containing
HFA134a + Ethanol after 30-31 days storage at 40° C. and 75% RH

| | | Weight | Amount of |
|---|---|---|---|
| | | loss | the leak |
| Example | Pouch content description | (mg) | adsorbed (%) |
| Example 1a | HFA134a + ethanol | 80 | -NA- |
| Example 2a | HFA134a + ethanol + silica | 92 | 74% |
| | gel | | |
| Example 3a | HFA134a + ethanol + Molecular | 79 | 51% |
| | Sieve 3A-EPG | | |
| Example 4a | HFA134a + ethanol + Molecular | 72 | 100% |
| | Sieve 13X-APG | | |
| Example 5a | HFA134a + ethanol + activated | 78 | 51% |
| | alumina A201 | | |
| Example 11a | HFA134a + ethanol + Molecular | 94 | 38% |
| | Sieve 4A | | |
| Example 12a | HFA134a + ethanol + Molecular | 71 | 100% |
| | Sieve 5A | | |
| Reference | HFA134a + ethanol + Molecular | 66 | 100% |
| Composition 1 | Sieve 13X-APG | | |
| Reference | HFA134a + ethanol | 76 | NA |
| Composition 2 | | | |

TABLE 2
Weight losses and leak adsorption for HFA134a/ethanol canisters
after 60 or 90 days storage at 40° C. and 75% RH

| | | | Weight | Amount of |
|---|---|---|---|---|
| | Pouch content | Days | loss | the leak |
| Example | description | storage | (mg) | adsorbed (%) |
| Example 1b | HFA134a + ethanol | 60 | 127 | -NA- |
| Example 2b | HFA134a + ethanol + | 60 | 111 | 61% |
| | silica gel | | | |
| Example 3b | HFA134a + ethanol + | 60 | 159 | 25% |
| | Molecular Sieve | | | |
| | 3A-EPG | | | |
| Example 4b | HFA134a + ethanol + | 60 | 109 | 100% |
| | Molecular Sieve | | | |
| | 13X-APG | | | |
| Example 5b | HFA134a + ethanol + | 60 | 164 | 14% |
| | activated alumina | | | |
| | A201 | | | |
| Example 11b | HFA134a + ethanol + | 90 | 247 | 39% |
| | Molecular Sieve 4A | | | |
| Example 12b | HFA134a + ethanol + | 90 | 259 | 100% |
| | Molecular Sieve 5A | | | |
| Reference | HFA134a + ethanol + | 90 | 143 | 100% |
| Composition 1 | Molecular Sieve | | | |
| | 13X-APG | | | |
| Reference | HFA134a + ethanol | 90 | 207 | -NA- |
| Composition 2 | | | | |

TABLE 3
Weight losses and leak adsorption for HFA134a/ethanol canisters
after 120 days storage at 40° C. and 75% RH

| | | | Amount of |
|---|---|---|---|
| | | | the leak |
| | | Weight | adsorbed |
| Example | Pouch content description | loss (mg) | (%) |
| Example 1c | HFA134a + ethanol | 312 | -NA- |
| Example 2c | HFA134a + ethanol + silica | 304 | 28% |
| | gel | | |
| Example 3c | HFA134a + ethanol + Molecular | 254 | 22% |
| | Sieve 3A-EPG | | |
| Example 4c | HFA134a + ethanol + Molecular | 312 | 100% |
| | Sieve 13X-APG | | |
| Example 5c | HFA134a + ethanol + activated | 336 | 19% |
| | alumina A201 | | |
| Example 11c | HFA134a + ethanol + Molecular | 132 | 36% |
| | Sieve 4A | | |
| Example 12c | HFA134a + ethanol + Molecular | 239 | 100% |
| | Sieve 5A | | |
| Reference | HFA134a + ethanol + Molecular | 153 | 100% |
| Composition 1 | Sieve 13X-APG | | |
| Reference | HFA134a + ethanol | 142 | -NA- |
| Composition 2 | | | |

TABLE 3a
Weight losses and leak adsorption for HFA134a/ethanol canisters
after 150 days storage at 40° C. and 75% RH

| | | Weight | Amount of the |
|---|---|---|---|
| | | loss | leak adsorbed |
| Example | Pouch content description | (mg) | (%) |
| Example 1d | HFA134a + ethanol | 259 | -NA- |
| Example 2d | HFA134a + ethanol + silica gel | 396 | 39% |
| Example 3d | HFA134a + ethanol + Molecular | 231 | 13% |
| | Sieve 3A-EPG | | |
| Example 4d | HFA134a + ethanol + Molecular | 253 | 100% |
| | Sieve 13X-APG | | |

TABLE 4
Weight losses and leak adsorption for canisters containing
HFA227 after 30-31 days storage at 40° C. and 75% RH

| | | | Amount of |
|---|---|---|---|
| | | Weight | HFA 227 |
| Example | Pouch content description | loss (mg) | adsorbed (%) |
| Example 6a | HFA227 | 30 | -NA- |
| Example 7a | HFA227 + silica gel | 28 | 94% |
| Example 8a | HFA227 + Molecular Sieve | 45 | 43% |
| | 3A-EPG | | |
| Example 9a | HFA227 + Molecular Sieve | 36 | 100% |
| | 13X-APG | | |
| Example 10a | HFA227 + activated | 27 | 80% |
| | alumina A201 | | |
| Example 13a | HFA227 + Molecular Sieve | 21 | 83% |
| | 4A | | |
| Example 14a | HFA227 + Molecular Sieve | 38 | 100% |
| | 5A | | |
| Reference | HFA227 + Molecular Sieve | 28 | 100% |
| Composition 1 | 13X-APG | | |
| Reference | HFA227 | 20 | -NA- |
| Composition 2 | | | |

TABLE 5
Weight losses for HFA227 canisters after 60 or 90 days storage at
40° C. and 75% RH

| | | | | Amount of |
|---|---|---|---|---|
| | | | Weight | the leak |
| | Pouch content | Days | loss | adsorbed |
| Example | description | Storage | (mg) | (%) |
| Example 6b | HFA227 | 60 | 36 | -NA- |
| Example 7b | HFA227 + silica gel | 60 | 45 | 87% |
| Example 8b | HFA227 + Molecular | 60 | 75 | 35% |
| | Sieve 3A-EPG | | | |
| Example 9b | HFA227 + Molecular | 60 | 37 | 100% |
| | Sieve 13X-APG | | | |
| Example 10b | HFA227 + activated | 60 | 59 | 60% |
| | alumina A201 | | | |
| Example 13b | HFA227 + Molecular | 90 | 84 | 92% |
| | Sieve 4A | | | |
| Example 14b | HFA227 + Molecular | 90 | 41 | 100% |
| | Sieve 5A | | | |
| Reference | HFA227 + Molecular | 90 | 94 | 100% |
| Composition 1 | Sieve 13X-AG | | | |
| Reference | HFA227 | 90 | 37 | -NA- |
| Composition 2 | | | | |

TABLE 6
Weight losses for HFA227 canisters after 120 days storage at 40° C.
and 75% RH

| | | | Amount of |
|---|---|---|---|
| | | | the leak |
| | | Weight | adsorbed |
| Example | Pouch content description | loss (mg) | (%) |
| Example 6c | HFA227 | 56 | -NA- |
| Example 7c | HFA227 + silica gel | 122 | 83% |
| Example 8c | HFA227 + Molecular Sieve 3A- | 99 | 50% |
| | EPG | | |
| Example 9c | HFA227 + Molecular Sieve | 63 | 100% |
| | 13X-APG | | |
| Example 10c | HFA227 + activated alumina | 43 | 9% |
| | A201 | | |
| Example 13c | HFA227 + Molecular Sieve 4A | 91 | 92% |
| Example 14c | HFA227 + Molecular Sieve 5A | 58 | 97% |
| Reference | HFA227 + Molecular Sieve | 111 | 100% |
| Composition 1 | 13X-AG | | |
| Reference | HFA227 | 110 | -NA- |
| Composition 2 | | | |

TABLE 7
Weight losses for HFA227 canisters after 150 days storage at 40° C.
and 75% RH

| | | | Amount of the |
|---|---|---|---|
| | | Weight loss | leak adsorbed |
| Example | Pouch content description | (mg) | (%) |
| Example 7d | HFA227 + silica gel | 140 | 34% |
| Example 8d | HFA227 + Molecular | 76 | 0% |
| | Sieve 3A-EPG | | |
| Example 9d | HFA227 + Molecular | 91 | 100% |
| | Sieve 13X-APG | | |

TABLE 8
Water capacity of the different desiccant used

| | | | | Acti- | Molec- | Molec- |
|---|---|---|---|---|---|---|
| | | Molecular | Molecular | vated | ular | ular |
| | Silica | Sieve | Sieve | alumina | Sieve | Sieve |
| | gel | 3A-EPG | 13X-APG | A-201 | 4A | 5A |
| Water | 30 | 20 | 24 | 40 | 17.5 | 23 |
| capacity (%) | | | | | | |
| Amount of | 1.5 | 2.2 | 1.8 | 1.1 | 2.5 | 1.9 |
| desiccant | | | | | | |
| required to | | | | | | |
| absorb | | | | | | |
| 0.34 g | | | | | | |
| (plus an | | | | | | |
| excess | | | | | | |
| of 30% for | | | | | | |
| safety) (in g) | | | | | | |

### EXAMPLE 15

pMDIs containing HFA 134a and ethanol in the ratio 88%:18% and formoterol fumarate as active ingredient in amount suitable to deliver 6 mcg for each actuation unpouched or pouched with the drug delivery assembly of the invention were stored in stressed conditions at 40° C./75% RH to investigate the chemical stability of the drug product. As a desiccant the molecular sieve 13X-APG has been used.

Degradation products and water content were periodically checked. In Table 9 the results after 6 months storage are reported.

TABLE 9
Degradation products and water content of pressurized metered
dose inhalers (pMDIs) containing formoterol fumarate
(6 μg/dose) in solution in HFA 134a and ethanol 88:12%
(w/w) stored at 40° C./75% RH in pouches with and without
molecular sieve 13X in comparison with unpouched pMDIs

| | | | 1.5 | 3 | 6 |
|---|---|---|---|---|---|
| Test | | Start | months | months | months |
| Unpouched | Degradation | 0.65 | 1.48 | 3.79 | 9.05 |
| | products/ | | | | |
| | Related | | | | |
| | substances (%) | | | | |
| | Water content | 1050 | 1378 | 1998 | 3275 |
| | (ppm) | | | | |
| Pouched | Degradation | 0.65 | 1.41 | 3.47 | 7.86 |
| | products/ | | | | |
| | Related | | | | |
| | substances (%) | | | | |
| | Water content | 929 | 924 | 864 | 1222 |
| | (ppm) | | | | |
| Pouched with | Degradation | 0.65 | 1.50 | 3.25 | 6.96 |
| desiccant | products/ | | | | |
| (molecular sieve | Related | | | | |
| 13X) | substances (%) | | | | |
| | Water content | 1025 | 823 | 743 | 658 |
| | (ppm) | | | | |

1. Drug delivery assembly comprising:
a pressurised container holding a drug formulation with a propellant;
a sealed enclosure which surrounds the container and which is made of a moisture impermeable or substantially moisture impermeable material; and
a gas adsorbing material within the enclosure,
wherein the gas adsorbing material is a microporous zeolite or molecular sieve having a pore opening size comprised between 4 Å and 20 Å.

2. Drug delivery assembly according to claim 1 wherein the pore opening size is comprised between 5 Å and 20 Å.

3. Drug delivery assembly according to claims 1 and 2 wherein the pore opening size is comprised between 8 Å and 15 Å.

4. Drug delivery assembly according to any preceding claim wherein the enclosure is flexible.

5. Drug delivery assembly according to any preceding claim wherein the propellant is a hydrofluoroalkane selected from 1,1,1,2-tetrafluoroethane (HFA134a) and 1,1,1,2,3,3,3-heptafluoropropane (HFA227) and their mixtures.

6. Drug delivery assembly according to any preceding claim wherein the drug formulation contains a co-solvent.

7. Drug delivery assembly according to any preceding claim wherein the co-solvent is ethanol.

8. Drug delivery assembly according to any preceding claim wherein the active ingredient in the drug formulation is formoterol, its enantiomer or diastereoisomer, salts or solvates thereof.
